(19) United States
(12) Patent Application Publication
(10) Pub. No.: US 2018/0206789 A1
(43) Pub. Date: Jul. 26, 2018

(54) EXTREMITY CUFF SUCH AS A FINGER CUFF, A METHOD AND A COMPUTER PROGRAM PRODUCT

(71) Applicant: Edwards Lifesciences Corporation, Irvine, CA (US)

(72) Inventors: Peiyuan Li, Amsterdam (NL); Olaf Schraa, Amsterdam (NL); Geertruida Lucretia van der Sar, Alphen aan den Rijn (NL)

(73) Assignee: Edwards Lifesciences Corporation, Irvine, CA (US)

(21) Appl. No.: 15/873,171

(22) Filed: Jan. 17, 2018

Related U.S. Application Data
(60) Provisional application No. 62/449,923, filed on Jan. 24, 2017.

(51) Int. Cl. A61B 5/00; A61B 5/026; A61B 5/022; A61B 5/0225; A61B 5/0295

(52) U.S. Cl. CPC A61B 5/6826; A61B 5/0261; A61B 5/0295; A61B 5/0225; A61B 5/02233

(57) ABSTRACT

The invention relates to an extremity cuff such as a finger cuff for determining a physiological parameter. The extremity cuff comprises an extremity module for releasably surrounding an extremity portion in a circumferential direction. The extremity module includes a bladder system for exerting a pressure on extremity tissue. Further, the finger module includes a photoplethysmograph system for performing a radiation measurement on extremity tissue. The bladder system includes multiple bladder volumes that are expandable and retractable independently of each other.

The invention relates to an extremity cuff such as a finger cuff for determining a physiological parameter, comprising an extremity module for releasably surrounding an extremity portion in a circumferential direction, the extremity module including a bladder system for exerting a pressure on extremity tissue and a photoplethysmograph system for performing a radiation measurement on extremity tissue.

Such a cuff determining a physiological parameter in tissue of an extremity of a human or animal body is e.g. known as a finger cuff disclosed in European patent publication EP 2 182 839 B1. The photoplethysmograph system and the bladder system may be used for determining, in non-invasive way, the arterial pressure waveform. Although the disclosed finger cuff functions properly there is an ongoing need for improvement.

It is an object of the invention to provide an extremity cuff according to the preamble having an increased measurement accuracy. Thereto, an extremity cuff according to the preamble is provided, wherein the bladder system includes multiple bladder volumes that are expandable and retractable independently of each other.

By providing bladder volume that may expand and retract independently of each other, a non-uniform pressure profile can be applied to the extremity of the human body, thereby getter better control of the operational behavior of the extremity cuff leading to improved measurement accuracy.

Further, a simplified product design can be obtained while miniaturizing system components and reducing a set of sensor sizes as the cuff is useful for a wide range of extremity sizes, i.e. one size fits all. Further, the cuff is easy to use and flexible in design enabling other opportunities for product design. The cuff can be manufactured with reduced costs and has an increased comfortability level for a patient.

By arranging the bladder volumes on separate circumferential portions of the extremity module, specific pressure profiles or functions can be applied. As an example, a first bladder volume can then be used for pressure measurement purposes, while a second bladder volume can be used to compensate for variations in the finger shape, size and/or volume change caused by a sequence of pressure measurements over time. Further, a particular bladder volume can be used to increase the comfort of the user bearing the finger cuff and/or to stabilize the measurement.

Alternatively, bladder volumes can be arranged on common circumferential portions of the extremity module and/or may partially or completely overlap each other.

In an advantageous manner a first bladder volume is filled with a liquid and a second bladder volume is filled with a gas. A liquid filled bladder volume can e.g. be used for static or quasi-static compensation, e.g. for the purpose of compensating for variations in the finger shape, size and/or volume change caused by a sequence of pressure measurements over time, and/or for increasing the comfortability for the user and/or for stabilizing the measurement.

A gas filled bladder volume is preferably used for pressure measurements, having the advantage of low energy consumption and quick response.

The invention also relates to a method.

Further, the invention relates to a computer program product. A computer program product may comprise a set of computer executable instructions stored on a data carrier, such as but not limited to a flash memory, a CD or a DVD. The set of computer executable instructions, which allow a programmable computer to carry out the method as defined above, may also be available for downloading from a remote server, for example via the Internet, e.g. as an app.

Other advantageous embodiments according to the invention are described in the following claims.

By way of example only, embodiments of the present invention will now be described with reference to the accompanying figures in which

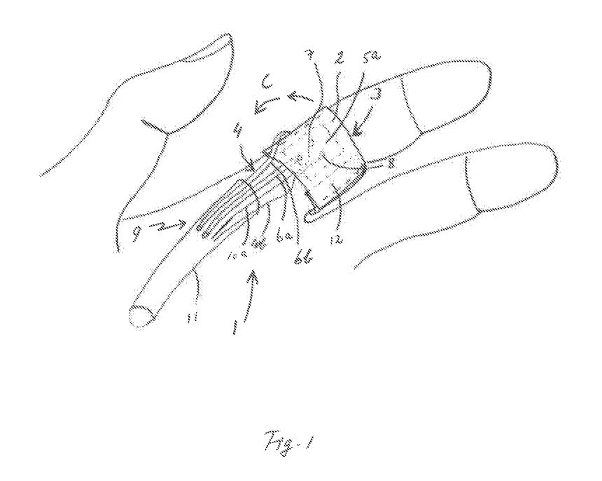

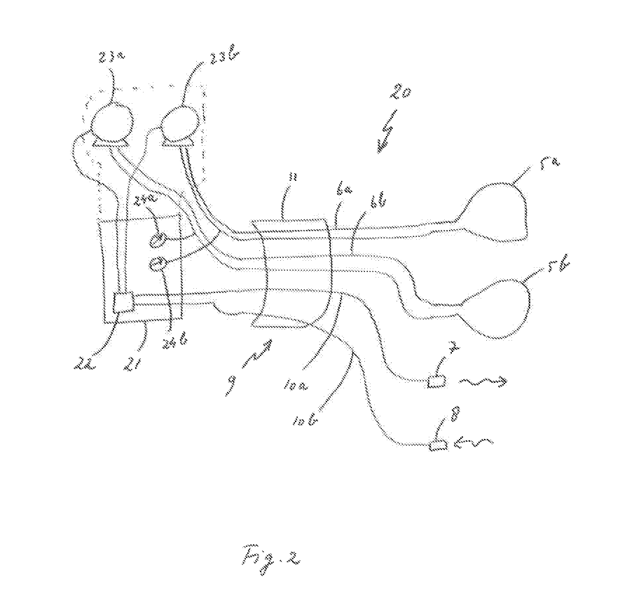

FIG. 1 shows a schematic perspective view of a first embodiment of a finger cuff according to the invention;

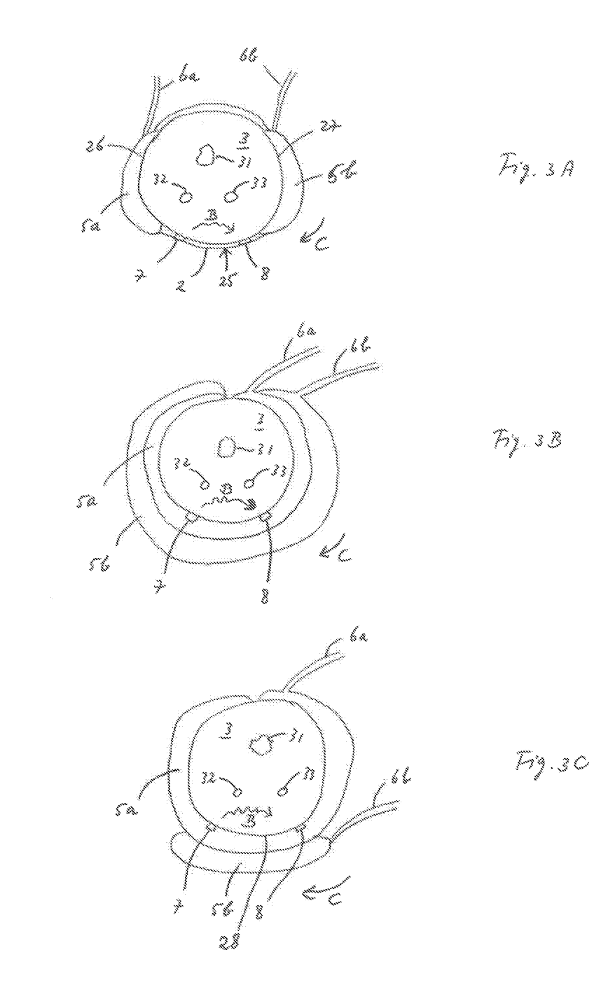

FIG. 2 shows a schematic system view of a cuff measurement system according to the invention;

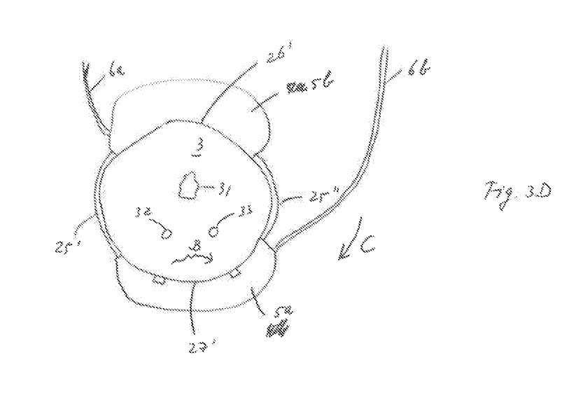

FIG. 3a shows a schematic cross sectional view of a second embodiment of a finger cuff according to the invention;

FIG. 3b shows a schematic cross sectional view of a third embodiment of a finger cuff according to the invention;

FIG. 3c shows a schematic cross sectional view of a fourth embodiment of a finger cuff according to the invention;

FIG. 3d shows a schematic cross sectional view of a fifth embodiment of a finger cuff according to the invention, and

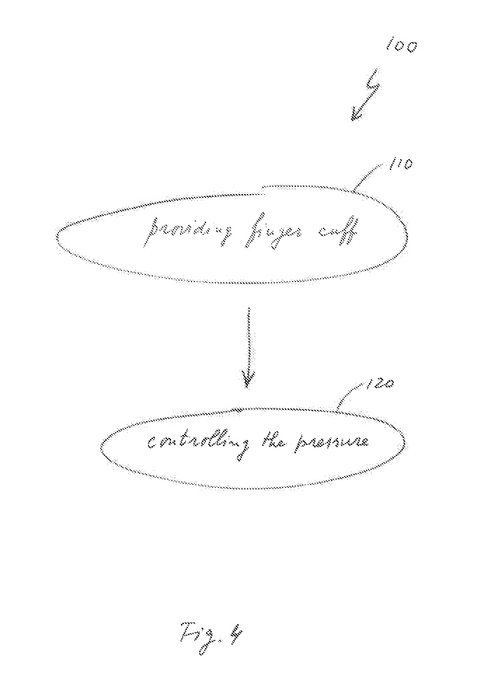

FIG. 4 shows a flow chart of a method according to the invention.

The figures merely illustrate preferred embodiments according to the invention. In the figures, the same reference numbers refer to equal or corresponding parts.

FIG. 1 shows a schematic perspective view of a first embodiment of a finger cuff 1 according to the invention. The finger cuff 1 is adapted for determining a physiological parameter, e.g. for measuring an arterial pressure waveform. Further, a plethysmogram and blood pressure data may be used for determining in a non-invasive way further information such as cardiac output.

The finger cuff 1 comprises a finger module 2 that releasably surrounds a finger portion 3 in a circumferential direction C. In FIG. 1 the finger module 2 surrounds the proximal phalanx. However, the finger module 2 can, alternatively, surround another phalanx, e.g. the middle phalanx of the finger. The finger module 2 includes a bladder system 4 that is arranged for exerting a pressure on finger tissue in the finger portion 3. Further, the finger module 2 includes a photoplethysmograph system for performing a radiation measurement on finger tissue in the finger portion 3. In the shown embodiment, circumferential end portions of the finger module 2 are connected to each other, e.g. using loop and hook material 12 or other fastening means, for surrounding the finger portion 3.

It is noted that, as indicated above, the finger module 2 can be wrapped around the finger enclosing the finger portion 3 in the circumferential direction C. However, the finger module 2 can be localized in an alternative way, e.g. using a configuration that is arranged for clamping or squeezing. Further, the finger module 2 may include a shrinking wall that first receives the finger portion and then shrinks to surround and clamp said finger portion. Further, the finger module 2 may include two module elements that are moveable relative to each other for exerting a squeezing force on the finger portion. In this context it is noted that the finger module may completely enclose the finger portion in the circumferential direction C or partially surround said finger portion in said circumferential direction C.

The bladder system 4 includes a first bladder volume 5a that is connected with a first fluid pressure line 6a feeding said first bladder volume 5a. Similarly, the bladder system 4 includes a second bladder volume arranged at the opposite side of the finger (not shown in FIG. 1), the second bladder volume being connected with a second fluid pressure line 6b. During use of the finger cuff, the pressure in the first and second bladder volume 5 can be monitored and controlled. The first and second fluid pressure lines 6a,b enable a suitable inflation and deflation of the respective inflatable bladder volumes 5, independent of each other.

Generally, the first and second bladder volume 5a-b may expand and contract in a controllable manner.

The bladder volume 5 has walls optionally formed from a thin flexible, translucent material. As an example, the bladder volume 5 is manufactured from two strips of film being heat sealed together about their periphery thereby forming a cavity. The cuff 1 is arranged to be fit about a person's finger. For this purpose the inflatable bladder volume 5 can be provided with a back-layer facing outwardly, away from finger tissue and a top-layer facing the tissue and being conceived to be brought into contact with the tissue.

In principle, the bladder volume 5 can be implemented in an alternative manner, e.g. as a body that expands and retracts responsive to electrical signals, the body including for example piezo material.

The photoplethysmograph system includes an emitter 7 for emitting a radiation in a direction of finger tissue in the finger portion 3 and a detector 8 for detecting radiation from said finger tissue. Preferably, the emitter 7 is implemented as a LED and the detector 8 is implemented as a photo diode.

In the inflatable bladder volume 5 openings may be provided for mounting therein the emitter 7 and detector 8. The inflatable bladder volume 5 can further be mounted on a flexible printed circuit that is used to feed electrical components of the cuff, including the emitter 7 and the detector 8 and to transmit measurement signals from the finger module 2 to a data processing unit for processing measurement data.

Photoplethysmograph systems are known per se. An operational principle of the photoplethysmograph system is based on the fact that with each cardiac cycle the heart pumps blood to the periphery of the body.

A change in volume of the arteries or arterioles caused by the pressure pulse of the systolic wave is detected by illuminating the skin with a suitable radiation, such as light emitted from the LED 7 and then measuring the amount of light either transmitted or reflected by tissue to the detector 8. Each cardiac cycle appears as a peak in a signal from the photoplethysmograph system. The shape of a signal waveform from the photoplethysmograph system differs from subject to subject, and varies with a location and a manner in which the cuff is attached to the tissue.

The radiation emitted by the emitter 7 and detected by the detector 8 can be inside the optic spectrum, such as red or other visible light, or outside the optic spectrum such as infrared IR or deep infrared radiation.

The finger cuff 1 shown in FIG. 1 further comprises an intermediate module 9 operatively connected to the finger module 2. The intermediate module 9 includes the two fluid pressure lines 6a-b that are operatively connected to the multiple bladder volumes 5, respectively. Further, the intermediate module 9 includes electrical communication circuitry 10 connected to the photoplethysmograph system and to a data unit described below. The fluid pressure lines 6 and electrical communication circuitry 10 are preferably encased in a flexible outer cable 11 to ensure safe and reliable operation of the finger cuff 1.

FIG. 2 shows a schematic system view of a cuff measurement system 20 according to the invention. The cuff measurement system 20 comprises the finger cuff 1 described above referring to FIG. 1 and a control unit 21 for controlling the photoplethysmograph system via the electrical communication circuitry 10. In the shown embodiment, the electrical communication circuitry 10 includes a control line 10a connected to the emitter 7 for controlling a light emitting process performed by the emitter 7, and a data line 10b connected to the detector 8 for transmitting light detection data from the detector 8. The control unit 21 is further arranged for controlling a process of regulating the pressure in the independently inflatable first and second bladder volumes 5a-b.

The process of inflating and deflating the first bladder volume 5a is independent of the process of inflating and deflating the second bladder volume 5b. As an example, the first bladder volume 5a can be inflated while the second bladder volume 5b is kept at a constant pressure, and vice versa.

The control unit 21 includes a data processing unit 22 for processing measurement data transmitted from the detector 8. Thereto, the electrical communication circuitry 10 is connected to the data processing unit 22. Further, the control unit 21 includes pump units 23a-b for pressurizing and depressurizing the fluid pressure lines 6a-b to inflate and deflate the corresponding bladder volumes 5a-b. In principle, the control unit 21 may further include valves for de-pressuring the fluid pressure lines 6a-b. Also, the control unit 21 includes pressure sensors 24a-b for measuring the pressure in the fluid pressure lines 6a-b.

It is noted that the cuff measurement system 20 generally includes further modules and connections, e.g. for providing electrical energy to active electrical components including the emitter 7 and the detector 8 of the photoplethysmograph system.

FIG. 3a shows a schematic cross sectional view of a second embodiment of the finger cuff 1. The finger portion 3 surrounded by the finger module 2 includes a bone 31 and two arteries 32, 33. The two bladder volumes 5a-b are located opposite to each other relative to the finger portion 3, on separate circumferential portions 26, 27 of the finger module 2. Contrary to the first embodiment of the finger cuff 1 shown in FIG. 1, the emitter 7 and detector 8 are not arranged in a bladder volume 5a,b but in an intermediate portion 25 of the finger module 2. During use of the finger cuff 1, the emitter 7 transmits a radiation beam B into the finger tissue and the detector 8 detects a reflected, refracted and/or transmitted radiation signal.

In the embodiment shown in FIG. 3a, the two bladder volumes 5a-b are located to the left and to the right of the finger arteries 32, 33. However, in an alternative embodiment as shown in FIG. 3d, the first bladder volume 5a is located below the finger arteries 32, 33, while the second bladder volume 5b is located opposite to the first bladder volume 5a, i.e. above the bone 31 in the finger portion 3.

The first bladder volume 5a, below the finger arteries 32, 33, can then be used for pressure measurement purposes, while the second bladder volume 5b, above the bone 31, can be used to compensate for variations in the finger shape, size and/or volume change caused by a sequence of pressure measurements over time. Further, the second bladder volume 5b can be used to increase the comfort of the user bearing the finger cuff 1. In addition, the second bladder volume 5b can be used to stabilize the measurement.

FIG. 3b shows a schematic cross sectional view of a third embodiment of a finger cuff 1 according to the invention. Here, the first and second bladder volume 5a-b are arranged on a common circumferential portion of the finger module 2, viz. almost along the entire circumferential contour around the finger portion 3. The bladder volumes almost or mainly completely overlap in the circumferential direction C.

FIG. 3c shows a schematic cross sectional view of a fourth embodiment of a finger cuff according to the invention. Again, the first and second bladder volume 5a-b are arranged on a common circumferential portion of the finger module 2, i.e. the bladder volumes 5a-b have a circumferential portion 28 in common. The first and second bladder volumes 5a-b overlap partially. The first bladder volume 5a almost completely surrounds the finger portion 3 while the second bladder volume 5b is located at the above-mentioned common circumferential portion 28.

FIG. 3d shows a schematic cross sectional view of a fifth embodiment of a finger cuff according to the invention as indicated above as an alternative to the embodiment shown in FIG. 3a. Referring to the second embodiment shown in FIG. 3a, the two bladder volumes 5a,b are again located on separate circumferential positions, opposite to each other. However, in the fifth embodiment the separate circumferential positions 26′, 27′ are now at the top and bottom side of the finger portion 3, not at the left and right side of the finger portion 3 as is the case in FIG. 3a. Intermediate portions 25′, 25″ of the finger module 2 are located between the two bladder volumes 5a,b, at the right and left side of the finger portion 3.

It is noted that, generally, the location of the emitter 7 and the detector 8 may deviate. As an example, the emitter 7 and the detector may be positioned opposite to each other, relative to the finger portion 3.

Generally, a fluid is used for inflating and deflating the bladder volumes 5. Preferably, the first bladder volume 5a is filled with a liquid and the second bladder volume 5b is filled with a gas such as air, or vice versa.

A liquid filled bladder volume can e.g. be used for static or quasi-static compensation, e.g. for the purpose of compensating for variations in the finger shape, size and/or volume change caused by a sequence of pressure measurements over time, and/or for increasing the comfortability for the user and/or for stabilizing the measurement.

A gas filled bladder volume is preferably used for pressure measurements, having the advantage of low energy consumption and quick response.

It is noted that, as an alternative, both bladder volumes 5a-b can be filled with a liquid or both can be filled with a gas, e.g. the same liquid or gas, respectively.

FIG. 4 shows a flow chart of a method according to the invention. The method 100 is used for determining a physiological parameter. The method 100 comprises a step of providing 110 an extremity cuff comprising an extremity module for releasably surrounding an extremity portion in a circumferential direction, the extremity module including a bladder system for exerting a pressure on extremity tissue and a photoplethysmograph system for performing a radiation measurement on extremity tissue, wherein the bladder system includes multiple bladder volumes that are expandable and retractable independently of each other, and a step of controlling 120 a process of regulating the size in the independently inflatable bladder volumes.

The method for determining a physiological parameter can be performed using dedicated hardware structures, such as FPGA and/or ASIC components. Otherwise, the method can also at least partially be performed using a computer program product comprising instructions for causing a processor of a computer system or a control unit to perform the above described step of the method according to the invention, or at least a sub-step thereof, e.g. a step of controlling a process of regulating the pressure in the independently inflatable bladder volumes.

All steps can in principle be performed on a single processor. However, it is noted that at least one sub-step can be performed on a separate processor. A processor can be loaded with a specific software module. Dedicated software modules can be provided.

The invention is not restricted to the embodiments described herein. It will be understood that many variants are possible.

As an example, the bladder system may include more than two bladder volumes that are inflatable independently of each other, e.g. three or four independently inflatable bladder volumes.

It is further noted that the cuff can be designed for measuring another extremity of a human or animal body, such as a toe or an earlobe. Generally, an extremity cuff such as a finger cuff is provided for determining a physiological parameter, comprising an extremity module for releasably surrounding an extremity portion in a circumferential direction, the extremity module including a bladder system for exerting a pressure on extremity tissue and a photoplethysmograph system for performing a radiation measurement on extremity tissue, wherein the bladder system includes multiple bladder volumes that are expandable and retractable independently of each other. In the described embodiments described above, the extremity cuff is a finger cuff, wherein the extremity module is a finger module surrounding a finger portion.

These and other embodiments will be apparent for the person skilled in the art and are considered to fall within the scope of the invention as defined in the following claims. For the purpose of clarity and a concise description features are described herein as part of the same or separate embodiments. However, it will be appreciated that the scope of the invention may include embodiments having combinations of all or some of the features described

1. An extremity cuff for determining a physiological parameter, comprising an extremity module for releasably surrounding an extremity portion in a circumferential direction, the extremity module including a bladder system for exerting a pressure on extremity tissue and a photoplethysmograph system for performing a radiation measurement on extremity tissue, wherein the bladder system includes multiple bladder volumes that are expandable and retractable independently of each other.

2. An extremity cuff according to claim 1, wherein the multiple bladder volumes are inflatable and deflatable independently of each other.

3. An extremity cuff according to claim 1, wherein the bladder volumes are arranged on separate circumferential portions of the extremity module.

4. An extremity cuff according to claim 1, wherein the bladder volumes are arranged on common circumferential portions of the extremity module.

5. An extremity cuff according to claim 1, wherein the bladder volumes partially or completely overlap in a circumferential direction of the extremity module.

6. An extremity cuff according claim 1, wherein a first bladder volume is filled with a liquid and a second bladder volume is filled with a gas.

7. An extremity cuff according to claim 1, wherein the photoplethysmograph system includes an emitter for emitting a radiation in a direction of finger tissue and a detector for detecting radiation from said finger tissue.

8. An extremity cuff according to claim 7, wherein the emitter is a LED and the detector is a photo diode.

9. An extremity cuff according to claim 1, further comprising an intermediate module operatively connected to the extremity module, the intermediate module including multiple fluid pressure lines operatively connected to the multiple bladder volumes, respectively.

10. An extremity cuff according to claim 9, wherein the intermediate module further includes electrical communication circuitry connected to the photoplethysmograph system.

11. A cuff measurement system, comprising an extremity cuff according to claim 10 and a control unit for controlling the photoplethysmograph system via the electrical communication circuitry and for controlling a process of regulating the pressure in the independently expandable and retractable bladder volumes.

12. A method for determining a physiological parameter, comprising the steps of:
providing an extremity cuff comprising a finger module for releasably surrounding an extremity portion in a circumferential direction, the extremity module including a bladder system for exerting a pressure on extremity tissue and a photoplethysmograph system for performing a radiation measurement on extremity tissue, wherein the bladder system includes multiple bladder volumes that are expandable and retractable independently of each other; and
controlling a process of regulating the size of the independently expandable and retractable bladder volumes.

13. A computer program product for determining a physiological parameter using an extremity cuff comprising an extremity module for releasably surrounding an extremity portion in a circumferential direction, the extremity module including a bladder system for exerting a pressure on extremity tissue and a photoplethysmograph system for performing a radiation measurement on extremity tissue, wherein the bladder system includes multiple bladder volumes that are expandable and retractable independently of each other, the computer program product comprising computer readable code for causing a processor to perform a step of controlling a process of regulating the size in independently expandable and retractable bladder volumes.
